(19) United States
(12) Patent Application Publication
(10) Pub. No.: US 2025/0322155 A1
(43) Pub. Date: Oct. 16, 2025

(54) SYSTEMS AND METHODS FOR DATA PARSING USING LARGE LANGUAGE MODEL

(71) Applicant: Paige.AI, Inc., New York, NY (US)

(72) Inventors: Ran GODRICH, New York, NY (US); Jan BERNHARD, Brooklyn, NY (US); Siqi LIU, New York, NY (US); Matthew LEE, London (GB); Razik YOUSFI, Brooklyn, NY (US); Donghun LEE, New York, NY (US); Alican BOZKURT, New York, NY (US)

(21) Appl. No.: 19/178,243

(22) Filed: Apr. 14, 2025

Related U.S. Application Data
(60) Provisional application No. 63/633,898, filed on Apr. 15, 2024.

(51) Int. Cl. G06F 40/226; G06F 40/40; G16H 15/00

(52) U.S. Cl. CPC G06F 40/226; G06F 40/40; G16H 15/00

(57) ABSTRACT

A computer-implemented method for report parsing using a large language model. The method includes receiving a plurality of raw reports; filtering the plurality of raw reports; extracting raw data from the plurality of raw reports; providing the extracted raw data to a large language model (LLM); providing a prompt to the LLM; receiving a response from the LLM, the response including data labels derived from the extracted raw data; validating the received response against the plurality of raw reports; and training a machine learning model using the received response.

### RELATED APPLICATION

This application claims priority to U.S. Provisional Application No. 63/633,898 filed on Apr. 15, 2024, which is incorporated herein in its entirety.

### TECHNICAL FIELD

Various embodiments of the present disclosure relate generally to the processing of reports and, more particularly, to utilizing artificial intelligence processes, such as large language models, to parse reports, extract raw data, and validate the extracted raw data.

### BACKGROUND

A large corpus of text reports, such as, for example, medical reports, may include a large volume of unstructured data, data in varying formats, data in formats that are not human-readable, or data in formats that are difficult for humans to read. Extracting relevant data from such reports, performing analysis on the data contained in the reports, or making other use of the data contained in the reports, may, therefore, be difficult or prohibitive for practical applications.

The present disclosure is directed to overcoming one or more of these above-referenced challenges.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a flowchart of a method of report parsing using large language models, according to one or more embodiments.

FIG. 2 depicts an exemplary process flow in a system for report parsing using large language models, according to one or more embodiments.

FIGS. 3A and 3B depict an exemplary system prompt presented to a large language model in a system for report parsing using large language models, according to one or more embodiments.

FIG. 4 depicts an exemplary computer device or system, in which embodiments of the present disclosure, or portions thereof, may be implemented.

FIG. 5 depicts a flow diagram for training a machine-learning model, in accordance with techniques discussed herein.

FIG. 6 depicts a flowchart of a method of report parsing using large language models, according to one or more embodiments.

FIGS. 7A, 7B, and 7C depict an exemplary data set presented to a large language model in a system for report parsing using large language models, according to one or more embodiments.

FIG. 8 depicts a functional block diagram of an exemplary computer device or system configured to executed techniques discussed herein.

Like reference numbers and designations in the various drawings indicate like elements.

### DETAILED DESCRIPTION OF EMBODIMENTS

Various embodiments of the present disclosure relate generally to enabling voice control of an interactive audiovisual environment, and monitoring user behavior to assess engagement.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

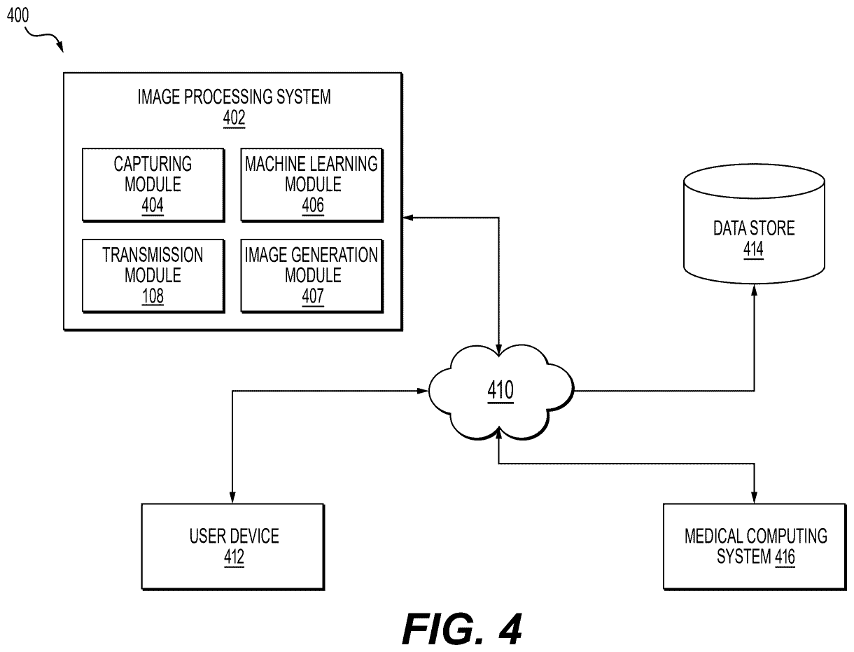

Any suitable system infrastructure may be put into place to allow user control of an interactive audiovisual environment, and engagement assessment. FIG. 4 and the following discussion provide a brief, general description of a suitable computing environment in which the present disclosure may be implemented. In one embodiment, any of the disclosed systems, methods, and/or graphical user interfaces may be executed by or implemented by a computing system consistent with or similar to that depicted in FIG. 4. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., electrically erasable programmable read-only memory (EEPROM) semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

As discussed above, it may be difficult or prohibitive in a practical application to extract relevant data from a large corpus of reports, perform analysis on the data contained in the reports, or make other use of the data contained in the reports. This may be due to the reports containing large volume of unstructured data, data in varying formats, data in formats that are not human-readable, or data in formats that are difficult for humans to read.

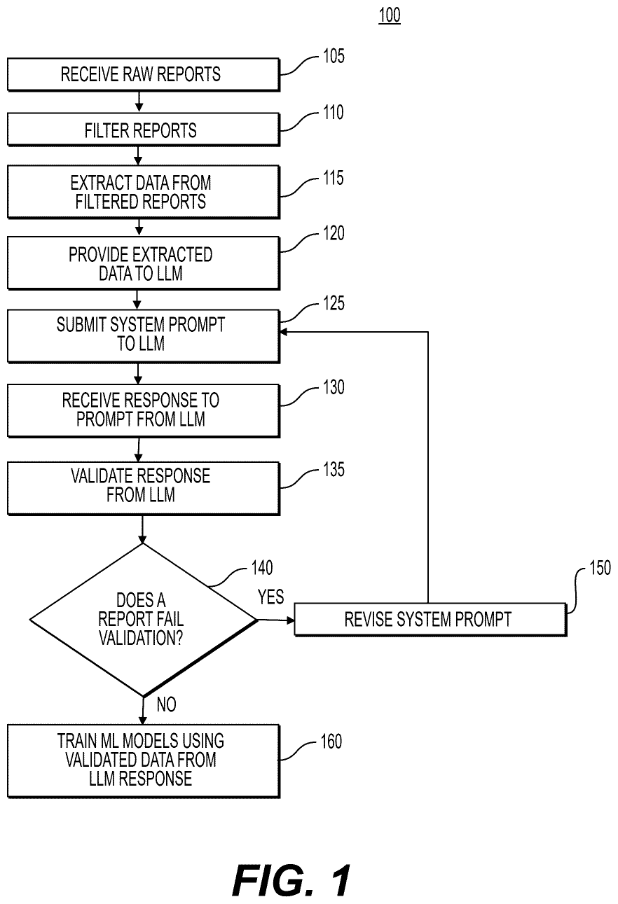

One or more embodiments of the present disclosure may provide systems and methods of report parsing using large language models (LLMs). As discussed below with respect to FIGS. 1-3, such systems and methods may include operations to filter raw reports, extract and/or unify data from the raw reports for processing by one or more LLMs, presenting a system prompt to the one or more LLMs, validation of the response(s) generated by the one or more LLMs, and use of the LLM response(s) for further analytics or other purposes. For example, the LLM response(s) may be used as training data for artificial intelligence processes, such as a machine learning model. These operations will be described in further detail below with respect to FIGS. 1-3.

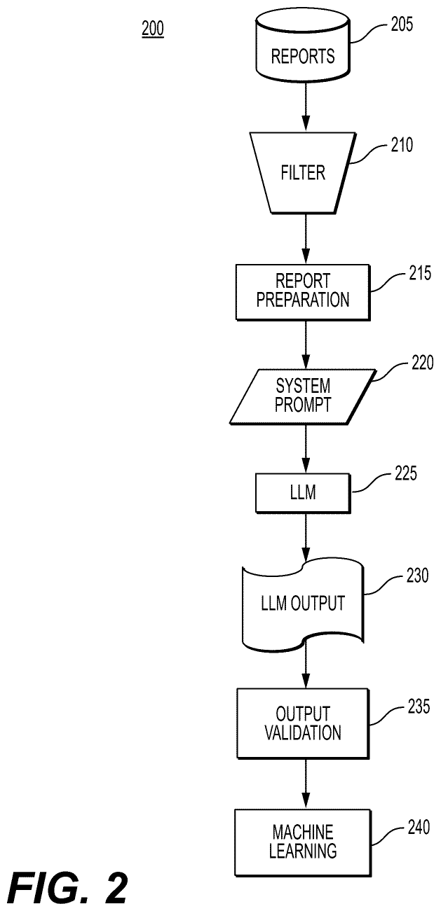

FIG. 1 depicts a flowchart of a method of report parsing using large language models, according to one or more embodiments, and FIG. 2 depicts an exemplary process flow in a system for report parsing using large language models, according to one or more embodiments.

As shown in FIG. 1, in operation 105, the method may receive raw reports, such as, for example, reports 205 depicted in FIG. 2. The raw reports may be received, for example, from a data store. The reports may be, for example, unstructured or semi-structured pathology reports associated with a patient which may be written by a pathologist, by a lab technician, such as, for example, in genetic testing, or may be automatically generated by a computer process. Each report may be a few words or several paragraphs, and may contain sensitive material, such as findings from immunohistochemistry (IHC) staining applied to pathology specimens to highlight protein expression. Such sensitive material may not be altered during processing by the disclosed operations. Ancillary studies and/or nuclear staining may also be considered IHC.

Each raw report may be, for example, an Integrated Mutation Profiling of Actionable Cancer Targets (IMPACT) genomic assay, a surgical report or diagnostic information (e.g., type(s) of cancer, grading, etc.), IHC staining report, or second opinions, molecular profiling reports, etc.

In operation 110, the method may filter the reports, such as by report filtering operation 210 depicted in FIG. 2. For example, the filtering may remove individual reports that do not contain task-specific information, contain duplicate information, do not contain the relevant image modalities and/or the relevant tissue types, contain corrupted text, etc. In another example, the filtering may occur on the basis of a tissue type. For example, it may be determined that the tissue type is from an identified region. The identified region may be associated with the patient. The identified region may be, for example, a stomach, colon, esophagus, or the like. In some examples, the tissue type may be a specimen sample collection, a biopsy, a resection, or the like. Not all reports in the corpus of reports 205 may be relevant to a particular inquiry (e.g., looking for specific biomarkers, looking for a specific type of carcinoma, etc.). Filtering operation 110 may determine a subset of reports relevant to the particular inquiry and only use relevant reports for further processing. Such filtering may further filter individual reports for length and consistency of the reports.

Such filtering may reduce the amount of data sent to an LLM for parsing and may, for example, reduce processing time and total costs when an LLM prompt is run over hundreds of thousands of reports. For example, the length of a prompt and the total length of the processed reports may be important for processing and cost efficiency. Such filtering may, thus, reduce the number of tokens processed by the final LLM and may be important for scaling the report parsing to very large numbers of reports (i.e., tens or hundreds of thousands of reports).

Filtering operation 210 may be implemented, for example, as an artificial intelligence operation (e.g., another LLM to parse each report and determine whether it matches a reference—i.e., whether the report is relevant), or may be implemented as a simpler deterministic expression (e.g., computer program code to look for certain keywords in each report and include or exclude the report based on the presence or absence of the keywords). In some embodiments, the reports may be manually filtered by human annotators.

The filtered set of raw reports may then be passed on to the next operation 115, in which a report preparation operation, such as by report preparation module 215 depicted in FIG. 2, may perform fine-grained extraction of raw data from the filtered reports. For example, report preparation module 215 may receive reports in a raw unstructured or semi-structured format, such as, for example, raw diagnostic and clinical information in human-readable or non-human readable formats such as, for example, java script object notation (JSON) format or other formats. The reports may contain semantic and numeric data, such as pseudocode, escape characters, ASCII, Unicode, URLs, formatting text, hypertext, abbreviations, CPT/ICD medical procedure codes, concatenations, acronyms, special characters, variable and function names from associated source code, etc. Report preparation operation 215 may convert the raw data into for example, binary label, numeric (e.g., integer or floating point notation) label, etc. For example, report preparation module 215 may extract important or valuable information (i.e., “ground truth”) from unstructured reports and generate labels such as, for example, binary labels, (e.g., 0/1, cancer/no cancer, etc.), continuous labels (e.g., regression analysis, etc.), categorical (e.g., cancer or disease type/subtype, etc.), a cancer subtype, presence or absence of a biomarker, etc.

Report preparation module 215 may, thus, unify raw reports 205, which may be in varying formats and may contain varying information. For example, reports generated through various clinical practices may present information in inconsistent formats, as the same concepts are expressed in different ways. Such reports may also be incomplete in that some information may and implications of stated information may not be stated explicitly but may be inferred from the stated information. In this way, report preparation module 215 may generate data from disparate reports that is consistent in format and contents.

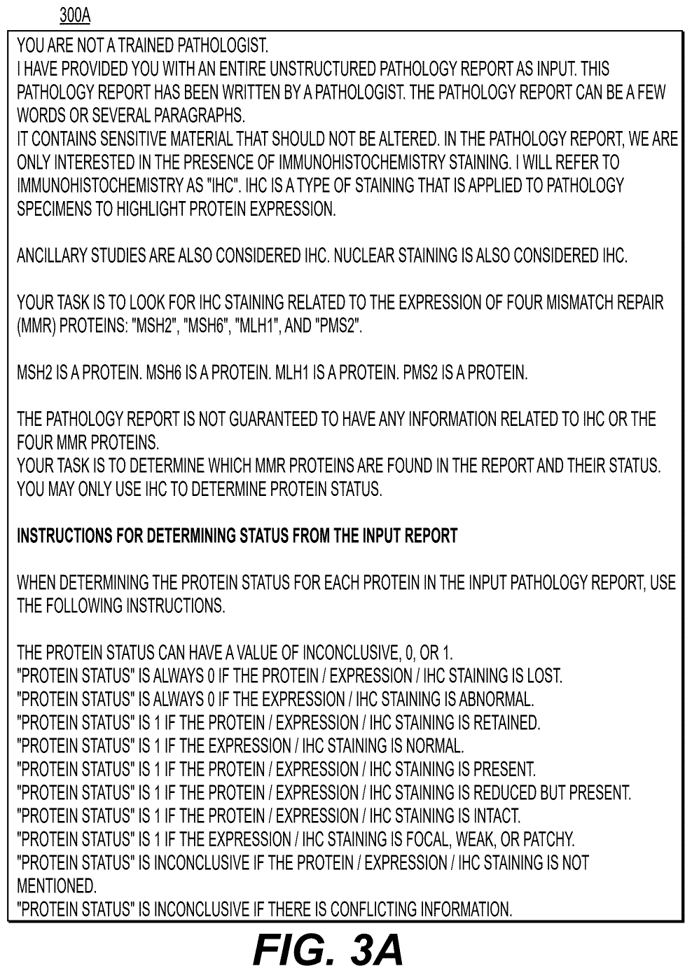

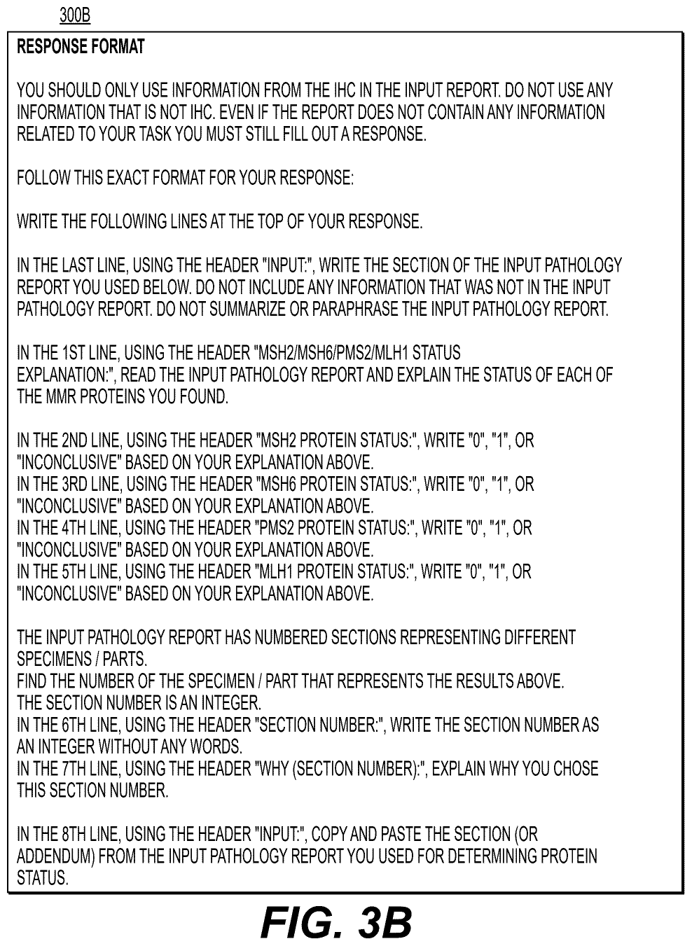

After raw report data 205 has been processed by report preparation module 215, in operation 120, the method may provide the extracted raw data to one or more large language models (LLMs), such as LLM 225 depicted in FIG. 2. In addition, in operation 125, the method may submit a system prompt, such as system prompt 220 depicted in FIG. 2 to the one or more LLMs 225. An example of such a prompt is prompt 300A-300B depicted in FIGS. 3A-3B. As shown in FIGS. 3A-3B, system prompt 220 may be engineered to ensure consistent and valid results from LLM 225. For example, system prompt 220 may include instructions to account for inconsistencies, incompleteness, differing formats, and anomalies in raw reports 205, ensure consistency in the response generated by LLM 225, and allow for validation of the response generated by LLM 225, as will be discussed in detail below.

In operation 130, the method may receive response(s) to the prompt from the one or more LLMs, such as LLM output 230 depicted in FIG. 2. LLM output 230 may include, for example, data extracted from raw reports 205 and rendered in a consistent normalized format. However, some characteristics of processing by an LLM may require that LLM output 230 be validated to ensure the accuracy and integrity of the data in LLM output 230. For example, some LLMs have been shown to “hallucinate” and present results that are not supported by underlying data. In addition, LLMs may be non-deterministic. That is, multiple executions of the same LLM based on the same corpus of raw reports and the same system prompt may result in different results or different expressions of the same results. For these reasons, the method may include validation of LLM output 230, such as by output validation module 230 depicted in FIG. 2.

In operation 135, the method may validate the response(s) received from the one or more LLMs, such as by output validation module 235 depicted in FIG. 2. System prompt 220 may include, for example, instructions to LLM 225 to return in LLM output 230 the extracted raw data and a portion of raw reports 205 that supports the extracted raw data. Output validation module 235 may, for example, compare the extracted raw data in LLM output 230 against the portion of raw reports 205 supporting the extracted raw data. In particular, output validation module 235 may, for example, look for a word-for-word match between the extracted raw data in LLM output 230 and the portion of raw reports 205 supporting the extracted raw data, but this method may be deemed as too strict in some applications. Alternatively, output validation module 235 may use vocabulary matching to validate extracted raw data for raw reports 205 that meets to predetermined threshold such as, for example, a 90% match between the extracted raw data in LLM output 230 and the portion of raw reports 205 supporting the extracted raw data. In other examples, the output validation module 235 may use keyword or key phrase matching to validate extracted raw data for raw reports 205 that matches predetermined key words or key phrases. Alternatively, output validation module 235 may use validated external data in comparison with the data in LLM output 230. Such a comparison may be performed for only a subset of the reports among raw reports 205.

In some embodiments, a validation process may validate and iterate on system prompt 220 itself by running system prompt 220 on a subset of raw reports 205 that have been reviewed by domain experts and have the target labels extracted from these reports in LLM output 230. To measure the effectiveness of system prompt 220 in extracting the desired information out of raw reports 205, a validation process may compute a mismatch rate in the extracted labels between the human domain experts and LLM output 230. After a combination of system prompt 220 and LLM 225 achieves a mismatch rate below an acceptable threshold, the combination may be used in an open system to extract training data from raw reports 205. Additionally, this process of tuning system prompt 220 may be used to determine an appropriate threshold for the verification feature described above.

In some embodiments, a validation process may use the nondeterministic nature of LLM 225 to validate a combination of system prompt 220 and LLM 225. For example, such a process may run the same report from raw reports 205 with the same system prompt 220 on the same LLM 225 multiple times. If this process provides different answers for the same request, the process may determine that the report is ambiguous and may mark the report for further processing. This type of validation may, thus, require a unanimous vote for labels associated with a given report across any number of iterations.

In addition, system prompt 220 may instruct LLM 225 to provide reasoning or analysis supporting the extracted raw data in LLM output 230. For example, system prompt 220 may instruct LLM 225 to use specific words or phrases in each report among raw reports 205. Such reasoning or analysis may be provided by LLM 225 either before or after the generation of LLM output 230.

To better ensure the accuracy and validity of LLM output 230, system prompt 220 may provide specific instructions to LLM 225 for performing data analysis. For example, system prompt 220 may provide specific operations to determine whether a label is valued as zero/one, yes/no, true/false, or inconclusive, etc. For example, system prompt 220 may provide specific vocabulary, keywords, key phrases, etc. that determine a binary value of a label. In addition, system prompt 220 may instruct LLM 225 that the absence of a label in a report implies that the value of the label should be false or zero. Alternatively, system prompt 220 may instruct LLM 225 that the absence of a label in a report implies that the value of the label should be inconclusive. If LLM 225 cannot determine conclusively that a label is present in the report, then the value of the label may be set as inconclusive. In an example, determination of protein expression in a report may be based on IHC staining results in the report, where the label value may the 1/true if the IHC stain result is positive, 0/false if the IHC stain result is negative, or inconclusive if no IHC stain result is provided.

System prompt 220 may also provide a context of the execution of the task-specific instructions, such as, for example: “You are a pathologist in training. You can't diagnose or interpret the findings presented in the report because you don't have the training to do so. However, you can summarize the findings to categorize them in correspondence with the information presented in the report. Remain factual to the reports contents in categorizations.”

In operation 140, the method may determine whether at least one report failed validation, such as by output validation module 235 depicted in FIG. 2. If at least one report failed validation, or a threshold number or percentage of the reports failed validation, then in operation 150, the method may revise the system prompt, and may then return to operation 125 for further processing. For example, output validation module 235 may re-run the analysis of only the reports that failed validation or may re-run the analysis of a larger or smaller subset of the reports, or may re-run the analysis of all reports. If no reports failed validation, or fewer than a threshold number or percentage of the reports failed validation, then the method may continue to operation 160. The selection of the threshold number or percentage of the reports failed validation may be based on a cost of re-running the reports, or may be based on a processing burden of re-running the reports, or both. In addition, output validation module 235 may determine whether the number of reports failing validation is increasing, i.e., that the report analysis is diverging. If it is determined that the report analysis is diverging, output validation module 235 may re-start the analysis from scratch, may go back to an earlier version of the system prompt, or may rely on manual intervention by a human operator.

In another example, the system prompt may be revised by enriching the context of specific keywords in a respective description field. For example, a weighting or value may be assigned to specific keywords. The value of the keyword may be compared to a threshold value associated with the specific keywords. If the value of the specific keyword is greater than the threshold value, then the system prompt may be revised with specific keyword. If the value of the specific keyword is less than the threshold value, then the system prompt may remain unchanged.

In operation 160, the method may train one or more artificial intelligence processes, such as, for example, one or more machine learning (ML) models 240 depicted in FIG. 2, training data 512, and/or machine learning module 406, using the validated data from the LLM response(s). For example, the validated data from the LLM response(s) may be provided to ML models 240 as structured ground truth with less user intervention than by conventional methods. That is, the LLM output 230 may be provided to ML models 240 directly as a data frame or other structured format in order to learn domain specific rules and/or subtleties. As an example, a model not trained with the validated data as structured ground truth may determine that a phrase such as “suspicious for adenocarcinoma” would indicate the presence of adenocarcinoma. Training the LLM using the validated data as structured ground truth may provide the model with domain specific context. In such an example, the phrase or word “suspicious” would indicate uncertainty in the context of the phrase, and would require the model to set the related diagnosis as neither present or absent. In other examples, a pre-trained LLM may be adjusted based on the validated data and then further trained by phrasing a learning objective for the model around the validated data for a given report. The learning objective may be based on a human reviewed structured ground truth data associated with a given raw pathology report.

The process described above, and depicted in FIGS. 2-3B, may be performed iteratively with corrections or modifications of the reports 205 and/or system prompt 220 such that all relevant clinical information or all training data from ML models is extracted at once, possibly independent of a specific ML training task. That is, LLM 225, or multiple LLMs 225, may be trained such that all possible labels may be extract from all reports 205 through a single iteration of processing by LLM(s) 225.

FIG. 4 depicts an exemplary environment 400 that may be utilized with techniques presented herein. One or more user device(s) 412 may communicate across an electronic network 410. The one or more user device(s) 412 may be associated with a user, e.g., a user that is viewing and/or interacting with a generated navigable three-dimensional image, an administrator of one or more components of environment 400, and/or the like. As will be discussed in further detail below, one or more computing system(s) 402 may communicate with one or more of the other components of the environment 400 across electronic network 410.

The user device(s) 412 may be configured to enable a user to access and/or interact with other systems in the environment 400. For example, the user device(s) 412 may each be a computer system such as, for example, a desktop/laptop computer, a mobile device, a tablet, an augmented/virtual/extended reality device, etc. In some embodiments, the user device(s) 412 may include one or more electronic application(s), e.g., a program, plugin, browser extension, etc., installed on a memory of the user device(s) 412. In some embodiments, the electronic application(s) may be associated with one or more of the other components in the environment 400. For example, the electronic application(s) may include one or more of system control software, system monitoring software, software development tools, etc.

In various embodiments, the environment 400 may include one or more data store 414 (e.g., database). The data store 414 may include a server system and/or a data storage system such as computer-readable memory such as a hard drive, flash drive, disk, etc. In some embodiments, the data store 414 includes and/or interacts with an application programming interface for exchanging data to other systems, e.g., one or more of the other components of the environment. The data store 414 may include and/or act as a repository or source for storing image data, whole slide images (WSI), a generated three-dimensional image, patient data, output data (e.g., from a machine-learning model), and the like (e.g., to be provided/transmitted to user device 412 or to/from any of the other components of environment 400).

In some embodiments, the components of the environment 400 are associated with a common entity, e.g., a service provider, an account provider, or the like. For example, in some embodiments, computing system 402, data store 414, and medical computing system 416 may be associated with a common entity. In some embodiments, one or more of the components of the environment may be associated with a different entity than another. For example, computing system 402 may be associated with a first entity (e.g., a service provider) while medical computing system 416 may be associated with a second entity (e.g., a medical institution or provider). The systems and devices of the environment 400 may communicate in any arrangement. As will be discussed herein, systems and/or devices of the environment 400 may communicate in order to one or more of generate, train, or use a machine-learning model to process imaging data, among other activities.

As discussed in further detail herein, the computing system(s) 402 may generate, store, train, communicate with, and/or use a machine-learning model(s), for example using machine learning module 406, configured to process imaging data. The computing system(s) 402 may include one or more machine-learning models and/or instructions associated with the machine-learning model, e.g., instructions for generating a machine-learning model, training the machine-learning model, using the machine-learning model etc. The computing system(s) 402 may include instructions for retrieving data, adjusting data, e.g., based on the output of the machine-learning model, and/or operating a display of the user device(s) 412 to output generated responses to input, e.g., as adjusted based on the machine-learning model. The computing system(s) 402 may include training data, e.g., image data, and may include ground truth, e.g., (i) training whole slide images and (ii) training three-dimensional images to generate a navigable three-dimensional image.

As depicted in FIG. 4, computing system(s) 402 may include capturing module 404. In various embodiments, capturing module 404 is configured to receive a plurality of whole slide images (WSI) associated with a tissue sample. The whole slide images and/or associated data may be gathered and/or compiled by the computing system 402 or using components separate from environment 400. In examples, capturing module 404 may receive the whole slide images from medical computing system 416 via network 410. Medical computing system 416 may be a user device associated with a medial institution, a medical imaging device, or the like. A medical imaging device implementing medical computing system 416 may include image processing system 402, or image processing system 402 may be a separate component from medical computing system 416. A plurality of images (e.g., digital or electronic image or a whole slide image (WSI)) may be received into electronic storage (e.g., cloud-based storage, hard disk, RAM, etc.) such as data store 414. Further, and in various embodiments, capturing module 404 may receive patient data. In examples, patient data may include medical records, demographic information, medical predispositions, diagnoses and the like. Such patient data may be received by capturing module 404 from data store 414, medical computing system 416, user device 412, or the like.

In example, such image data and patient data may be provided to one or more image processing machine-learning models. The one or more image processing machine-learning models may be implemented, generated, trained, or the like by machine-learning module 406. The one or more image processing machine-learning models may be trained based on training data that includes historical/genuine/prior patient tissue images and/or simulated/synthetic image data, historical or simulated patient data, and/or the like. Patient image processing techniques herein may use techniques described in U.S. application Ser. No. 17/313,617, which is incorporated by reference herein. Synthetic image generation may use techniques described in U.S. application Ser. No. 17/645,197, which is incorporated herein by reference. The training data may be used to train the image processing machine-learning models by modifying one or more weights, layers, synapses, biases, and/or the like of the image processing machine-learning models, in accordance with one or more machine-learning algorithms, as discussed herein. Alternatively, or in addition, such image data may be used to generate a three-dimensional image.

Computing system(s) 402 may also include image generation module 407. In various embodiments, image generation module 407 may be configured to generate a navigable three-dimensional image of a tissue sample based on an output of the one or more machine-learning models. In various embodiments, image generation module 407 may also be configured to generate an interactive display that incorporates the navigable three-dimensional image. In examples, the interactive display enables a user to navigate aspects of the three-dimensional image (e.g., zoom in/out, rotate, flip, view a cross-section, “peel back” layers of the three-dimensional image to view interior aspects, and the like). In further examples, the interactive display that incorporates the navigable three-dimensional image may be operable and/or configured to enable a user to navigate sample levels (e.g., tissue depths of the tissue sample associated with the image(s). Each level may be associated with a WSI.). In other various embodiments, image generation module 407 may be configured to generate a side-by-side display incorporating graphical representations of two or more images (e.g., whole slide images). In various additional embodiments, image generation module 407 may be configured to place a set of whole slide images in an order based an output of a machine-learning model, and may be further configured to “stitch” the whole slide images together based on the ordering.

As depicted in FIG. 4, computing system(s) 402 may also include transmission module 408. In various embodiments, transmission module 407 may be configured to transmit the interactive display, the side-by-side display, and/or the generated navigable three-dimensional image to a user interface, such as of user device 412. In further embodiments, transmission module 407 may be further configured to transmit the aforementioned to data store 414 (e.g., for storage or retention), or to medical computing system 416 (e.g., for storage, display, further processing, or the like).

As depicted in FIG. 4, environment 400 may also include electronic network 410. In various embodiments, the electronic network 410 may be a wide area network (“WAN”), a local area network (“LAN”), personal area network (“PAN”), or the like. In some embodiments, electronic network 410 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

Although depicted as separate components in FIG. 4, it should be understood that a component or portion of a component in the environment 400 may, in some embodiments, be integrated with or incorporated into one or more other components. In another example, the computing system 402 may be integrated in a data storage system. The data storage system may be configured to communicate and/or receive/send data across electronic network 410 to other components of environment 400. In some embodiments, operations or aspects of one or more of the components discussed above may be distributed amongst one or more other components. Any suitable arrangement and/or integration of the various systems and devices of the environment 400 may be used.

It should be understood that in various embodiments, various components of the environment 400 discussed above may execute instructions or perform acts including the acts discussed above. An act performed by a device may be considered to be performed by a processor, actuator, or the like associated with that device. Further, it should be understood that in various embodiments, various steps may be added, omitted, and/or rearranged in any suitable manner.

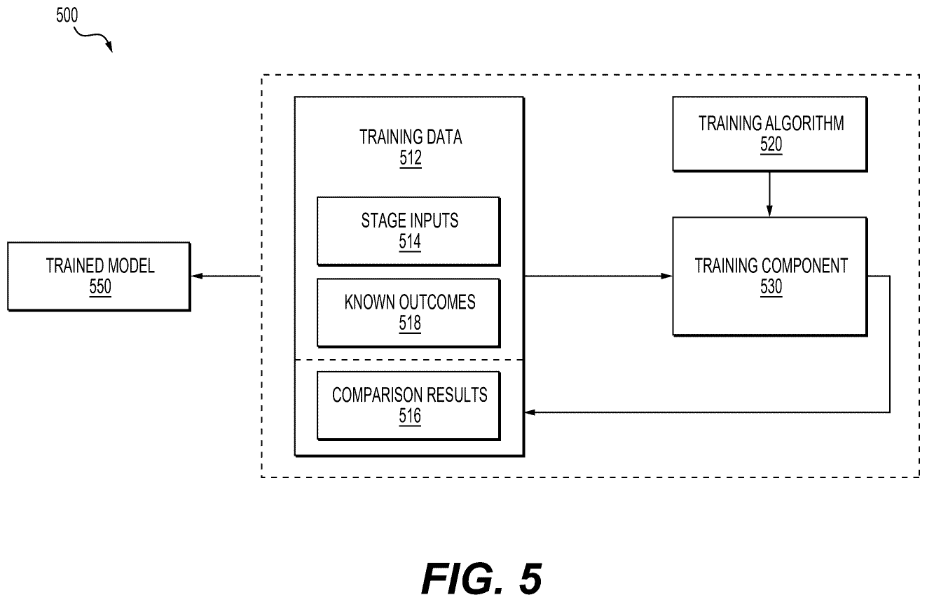

FIG. 5 depicts a flow diagram for training a machine-learning model. As shown in flow diagram 500 of FIG. 5, training data 512 may include one or more of stage inputs 514 and known outcomes 518 related to a machine-learning model to be trained. Diagram 500 may utilize modules that may be at least a portion of the functions of machine learning module 406. The stage inputs 514 may be from any applicable source including a component or set shown in the figures provided herein. The known outcomes 518 may be included for machine-learning models generated based on supervised or semi-supervised training. An unsupervised machine-learning model might not be trained using known outcomes 518. Known outcomes 518 may include known or desired outputs for future inputs similar to or in the same category as stage inputs 514 that do not have corresponding known outputs.

The training data 512 and a training algorithm 520 may be provided to a training component 530 that may apply the training data 512 to the training algorithm 520 to generate a trained machine-learning model 550. According to an implementation, the training component 530 may be provided comparison results 516 that compare a previous output of the corresponding machine-learning model to apply the previous result to re-train the machine-learning model. The comparison results 516 may be used by the training component 530 to update the corresponding machine-learning model. The training algorithm 520 may utilize machine-learning networks and/or models including, but not limited to a deep learning network such as Graph Neural Networks (GNN), Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 500 may be a trained machine-learning model 550, which may correspond to machine learning module 406, image generation module 407, and/or may be otherwise utilized by the image processing system 402.

A machine-learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine-learning model (e.g., a trained model) based on the training. Once trained, the machine-learning model may output machine-learning model outputs in accordance with the subject matter disclosed herein. According to an implementation, one or more machine-learning models disclosed herein may continuously be updated based on feedback associated with use or implementation of the machine-learning model outputs.

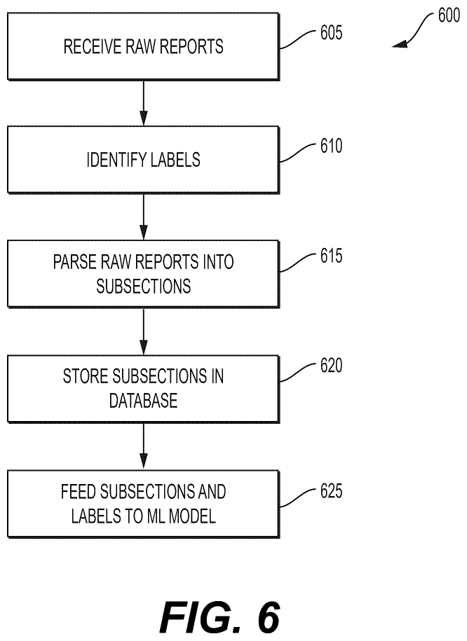

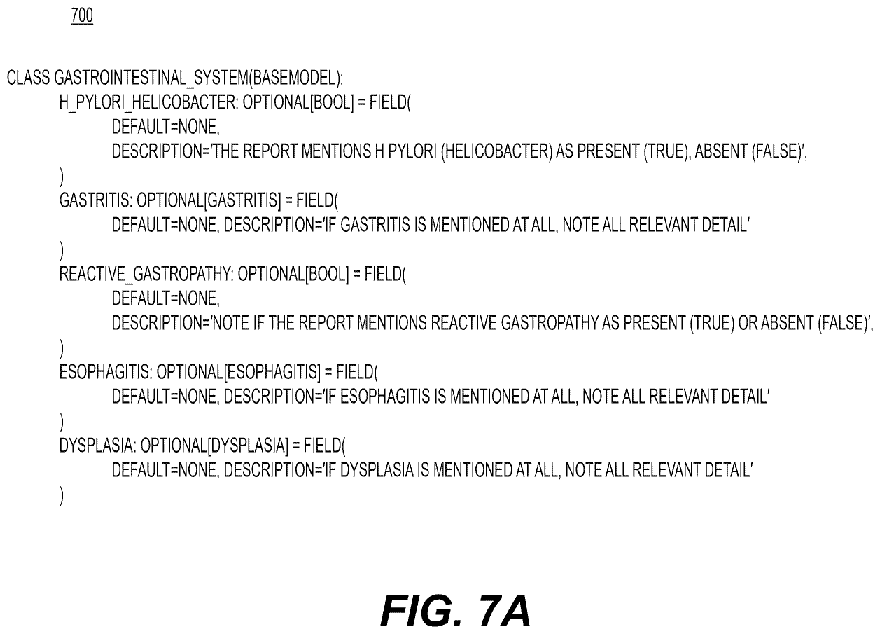

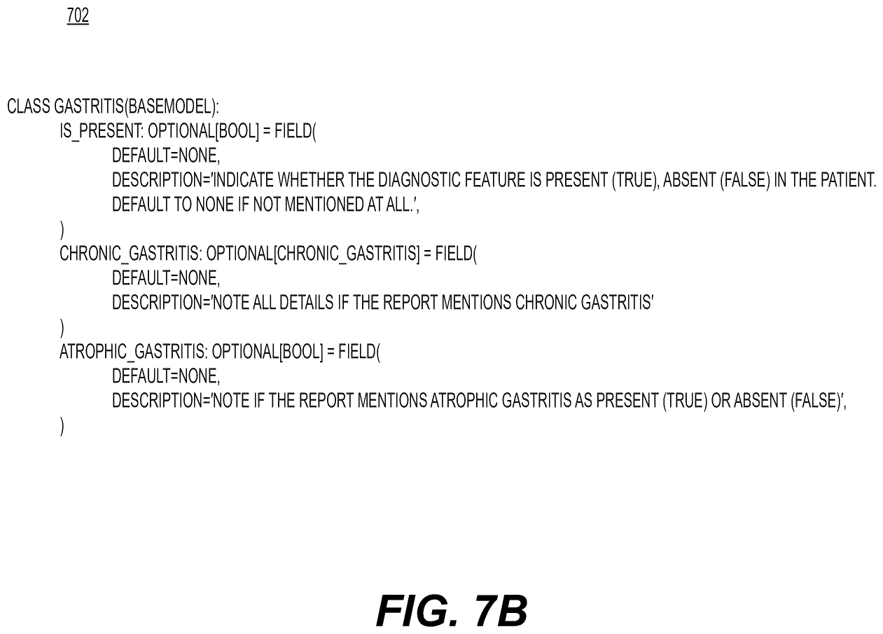

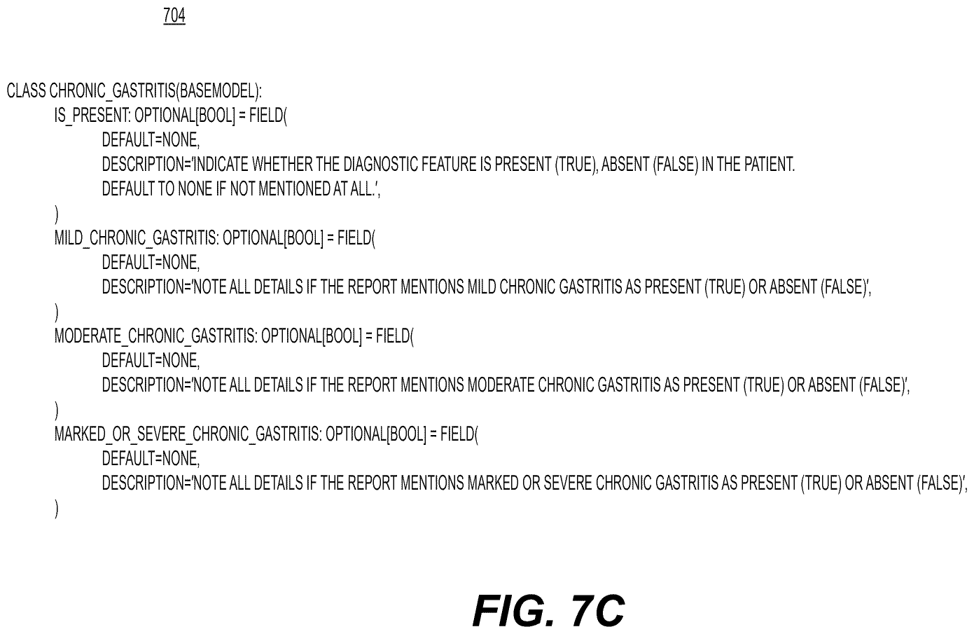

FIG. 6 depicts a flowchart of a method 600 of training ML models for report parsing using large language models, according to one or more embodiments, and FIGS. 7A-7C depicts exemplary nested sets of data in a system for training ML models for report parsing using large language models, according to one or more embodiments.

As shown in FIG. 6, in operation 605, the method may continuously receive raw reports, such as, for example, the reports 205 depicted in FIG. 2. In operation 610, the method may identify one or more labels associated with the reports. The one or more labels may be in a computer-readable format. For example, the one or more labels may be in a .csv file. In operation 615, the method may parse the raw reports into one or more subsections using an identified algorithm. For example, a graph-based algorithm may translate the one or more labels to a computer-readable medium and may generate one or more nested sets of data 700, 702, and 704 (shown in FIGS. 7A-7C) associated with the one or more labels. By generating the nested set of data, the LLM may be guided through extraction of the reports 205 and the nested set of data may reduce the number of required output tokens. Such guidance of the LLM may improve accuracy of the LLM.

In operation 620, the one or more subsections are stored in a datastore. In operation 625, the one or more subsections and the one or more labels are fed as input to a ML model.

It should be understood that aspects in this disclosure are exemplary only, and that other aspects may include various combinations of features from other aspects, as well as additional or fewer features.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

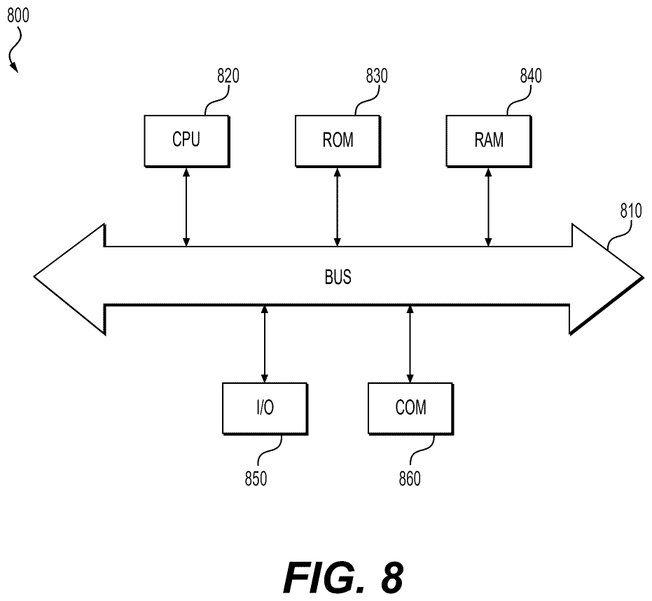

FIG. 8 depicts a high-level functional block diagram of an exemplary computer device or system, in which embodiments of the present disclosure, or portions thereof, may be implemented, e.g., as computer-readable code. Additionally, each of the exemplary computer servers, databases, user interfaces, modules, and methods described above with respect to FIGS. 1-3B and 6-7C can be implemented in environment 400 using hardware, software, firmware, tangible computer readable media having instructions stored thereon, or a combination thereof and may be implemented in one or more computer systems or other processing systems. Hardware, software, or any combination of such may implement each of the exemplary systems, user interfaces, and methods described above with respect to FIGS. 1-3B and 6-7C.

If programmable logic is used, such logic may be executed on a commercially available processing platform or a special purpose device. One of ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, at least one processor device and a memory may be used to implement the above-described embodiments. A processor device may be a single processor or a plurality of processors, or combinations thereof. Processor devices may have one or more processor “cores.”

Various embodiments of the present disclosure, as described above in the examples of FIGS. 1-3B and 6-7C, may be implemented using environment 400. After reading this description, it will become apparent to a person skilled in the relevant art how to implement embodiments of the present disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

As shown in FIG. 8, device 800 may include a central processing unit (CPU) 820. CPU 820 may be any type of processor device including, for example, any type of special purpose or a general-purpose microprocessor device. As will be appreciated by persons skilled in the relevant art, CPU 820 also may be a single processor in a multi-core/multiprocessor system, such system operating alone, or in a cluster of computing devices operating in a cluster or server farm. CPU 820 may be connected to a data communication infrastructure 810, for example, a bus, message queue, network, or multi-core message-passing scheme.

Device 800 also may include a main memory 840, for example, random access memory (RAM), and also may include a secondary memory 830. Secondary memory 830, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 830 may include other similar means for allowing computer programs or other instructions to be loaded into device 800. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 800.

Device 800 also may include a communications interface (“COM”) 860. Communications interface 860 allows software and data to be transferred between device 800 and external devices. Communications interface 860 may include a modem, a network interface (such as an Ethernet card), a communications port, a PCMCIA slot and card, or the like. Software and data transferred via communications interface 860 may be in the form of signals, which may be electronic, electromagnetic, optical, or other signals capable of being received by communications interface 860. These signals may be provided to communications interface 860 via a communications path of device 800, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 800 also may include input and output ports 850 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A computer-implemented method for report parsing using a large language model, the method comprising:
receiving a plurality of raw reports;
filtering the plurality of raw reports;
extracting raw data from the plurality of raw reports;
providing the extracted raw data to a large language model (LLM);
providing a prompt to the LLM;
receiving a response from the LLM, the response including data labels derived from the extracted raw data;
validating the received response against the plurality of raw reports; and
training a machine learning model using the received response.

2. The computer-implemented method of claim 1, further comprising, upon determining that one or more reports among the plurality of raw reports failed validation:
modifying the prompt or one or more reports among the plurality of raw reports; and
providing the modified prompt or the modified one or more reports to the LLM.

3. The computer-implemented method of claim 1, wherein determining that one or more reports among the plurality of raw reports failed validation further comprises comparing a number of the one or more reports that failed validation against a predetermined threshold.

4. The computer-implemented method of claim 1, further comprising, upon determining that a number of reports among the plurality of raw reports that failed validation has increased:
modifying the prompt to remove any modifications, or
modifying the prompt to match an intermediate version of the prompt, or
request manual intervention by a user.

5. The computer-implemented method of claim 1, wherein the plurality of raw reports includes one or more of: genomic assays, surgical reports, diagnostic information, histochemical stainings, or second opinions.

6. The computer-implemented method of claim 1, wherein the prompt includes instructions to cite a portion of an underlying raw report among the plurality of raw reports for each data label derived from the extracted raw data.

7. The computer-implemented method of claim 6, wherein validating the received response against the plurality of raw reports comprises comparing each derived data label against the cited portion of the underlying raw report.

8. The computer-implemented method of claim 1, wherein the prompt includes instructions for determining data labels.

9. The computer-implemented method of claim 1, wherein filtering the plurality of raw reports further comprises:
detecting keywords in each raw report among the plurality of raw reports, or
parsing each raw report among the plurality of raw reports using a second large language model (second LLM).

10. A system for report parsing using a large language model, the system comprising:
a data storage device storing instructions for report parsing using a large language model in an electronic storage medium; and
a processor configured to execute the instructions to perform operations comprising:
receiving a plurality of raw reports;
filtering the plurality of raw reports;
extracting raw data from the plurality of raw reports;
providing the extracted raw data to a large language model (LLM);
providing a prompt to the LLM;
receiving a response from the LLM, the response including data labels derived from the extracted raw data;
validating the received response against the plurality of raw reports; and
training a machine learning model using the received response.

11. The system of claim 10, wherein the operations further comprise, upon determining that one or more reports among the plurality of raw reports failed validation:
modifying the prompt or one or more reports among the plurality of raw reports; and
providing the modified prompt or the modified one or more reports to the LLM.

12. The system of claim 10, wherein determining that one or more reports among the plurality of raw reports failed validation further comprises comparing a number of the one or more reports that failed validation against a predetermined threshold.

13. The system of claim 10, wherein the operations further comprise, upon determining that a number of reports among the plurality of raw reports that failed validation has increased:
modifying the prompt to remove any modifications, or
modifying the prompt to match an intermediate version of the prompt, or
request manual intervention by a user.

14. The system of claim 10, wherein the prompt includes instructions to cite a portion of an underlying raw report among the plurality of raw reports for each data label derived from the extracted raw data.

15. The system of claim 14, wherein validating the received response against the plurality of raw reports comprises comparing each derived data label against the cited portion of the underlying raw report.

16. A non-transitory machine-readable medium storing instructions that, when executed by a computing system, causes the computing system to perform operations for report parsing using a large language model, the operations comprising:
receiving a plurality of raw reports;
filtering the plurality of raw reports;
extracting raw data from the plurality of raw reports;
providing the extracted raw data to a large language model (LLM);
providing a prompt to the LLM;
receiving a response from the LLM, the response including data labels derived from the extracted raw data;
validating the received response against the plurality of raw reports; and
training a machine learning model using the received response.

17. The non-transitory machine-readable medium of claim 16, the operations further comprising, upon determining that one or more reports among the plurality of raw reports failed validation:
modifying the prompt or one or more reports among the plurality of raw reports; and
providing the modified prompt or the modified one or more reports to the LLM.

18. The non-transitory machine-readable medium of claim 16, wherein determining that one or more reports among the plurality of raw reports failed validation further comprises comparing a number of the one or more reports that failed validation against a predetermined threshold.

19. The non-transitory machine-readable medium of claim 16, the operations further comprising, upon determining that a number of reports among the plurality of raw reports that failed validation has increased:
modifying the prompt to remove any modifications, or
modifying the prompt to match an intermediate version of the prompt, or
request manual intervention by a user.

20. The non-transitory machine-readable medium of claim 16, wherein the prompt includes instructions to cite a portion of an underlying raw report among the plurality of raw reports for each data label derived from the extracted raw data.
